(19) United States
(12) Patent Application Publication
(10) Pub. No.: US 2015/0272754 A1
(43) Pub. Date: Oct. 1, 2015

(54) DEVICE FOR DEPLOYING A COATED STENT IN THE CAROTID ARTERY

(71) Applicant: Zaza Alexandrovich Kavteladze, Moscow (RU)

(72) Inventor: Zaza Alexandrovich Kavteladze, Moscow (RU)

(21) Appl. No.: 14/436,683

(22) PCT Filed: Oct. 2, 2013
(86) PCT No.: PCT/RU2013/000872

§ 371 Date: Apr. 17, 2015

(30) Foreign Application Priority Data
Oct. 18, 2012 (RU) 2012144359

(51) Int. Cl. A61F 2/95; A61F 2/89

(52) U.S. Cl. CPC A61F 2/95; A61F 2/89; A61F 2002/9517

(57) ABSTRACT

The invention relates to medicine, in particular, to vascular and endovascular surgery. The objective of the invention is to provide accurate positioning of a coated stent in the carotid artery. The device is made in the form of a frame with an interior coating, the frame being made in the form of two rings interconnected by rods. A stopper limiting the introduction of the frame is fixed at one of the rings. The frame and the rings can have different shapes.

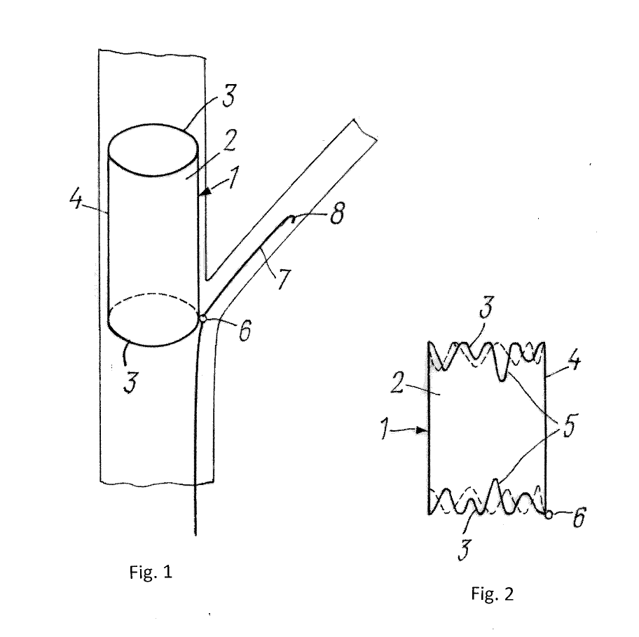

The invention relates to medicine, in particular, to vascular and endovascular surgery.

A carotid artery-implantable stent is known (US 380831, 1997, D24/155). A disadvantage of this stent is in a possibility of penetration of atherosclerotic plaques through the stent meshes, their possible subsequent detachment and entering brain blood vessels, which affects the efficacy of the surgery.

A coated carotid artery-implantable stent-graft covered with an exterior coating is known. The coating prevents atherosclerotic plaques from penetration through the stent meshes. However, the stent-graft has the following disadvantages: impossibility of accurate positioning of the stent-graft when introduced into the carotid artery due to uncontrolled self-expansion during the implantation when the stent-graft is pushed out from a delivery system, and due to a large size of the delivery device (see U.S. Pat. No. 5,628,788, A61F 2/06,1997).

The objective of the invention is to eliminate the above-indicated disadvantages and to develop a device providing accurate positioning of a coted stent in the carotid artery.

The objective is achieved by providing a device for positioning a stent in the carotid artery, the device being made in the form of a frame with an interior coating, the frame being made in the form of at least two rings interconnected by at least two rods, wherein the frame has a stopper limiting its introduction into the carotid artery and may have a conic or cylindrical shape. The rings of the frame may have a zigzag or U-like shape with uniform or nonuniform arms. The coating can be made of a resorbable material. The introduction-limiting stopper is made in the form of a loop and a tie rod interacting therewith, wherein the tie rod has a flexible end.

The figure shows a general view of the claimed invention.

The device for positioning of a coated stent in the carotid artery, according to the invention, is made of frame 1 having interior coating 2, the frame being made in the form of at least two rings 3 interconnected by at least two rods 4. Rings 3 have a zigzag or U-like shape with uniform or nonuniform arms 5. Stopper limiting the introduction of frame 1 into the carotid artery is fixed at one of rings 3, stopper being made in the form of loop 6 and tie rod 7 interacting therewith, wherein the tie rod has flexible end 8.

The device is used as follows:

Before positioning a coated stent, frame 1 with interior coating 2 is introduced into the carotid artery. The introduction of frame 1 is carried out by a delivery system (not shown) and is limited by loop 6 fixed at one of rings 3 and tie rod 7 with flexible end 8, wherein the latter ones rest against a branched region in the carotid artery, thus allowing maximal accuracy in positioning of frame 1. Rings 3 optimally self-lock to the arterial walls when pushed out from the delivery system. Selection of the shape (conic or cylindrical) of frame 1 depends on the pre-diagnostically determined anatomy of the carotid artery. The zigzag or U-like structure of the ring with uniform or nonuniform arms 5 makes it possible to use frame 1 depending on the anatomy of the carotid artery. Then a stent (not shown) is positioned inside frame 1. Coating 2 made of a resorbable material allows avoiding the development of pressure ulcers inside the lumen of vessels.

Thus, disposal of a stent-graft inside frame 1 provides double isolation preventing atherosclerotic plaques from penetration, and accurate positioning of the stent in the carotid artery, thus increasing the efficacy and safety of stenting.

1. A device for positioning a coated stent in the carotid artery, characterized in that the device is made of a frame with an interior coating, the frame being made in the form of at least two rings interconnected by at least two rods, wherein a stopper limiting the introduction of the frame into the carotid artery is fixed at one of the rings.

2. The device of claim 1 characterized in that the introduction-limiting stopper is made in the form of a loop and a tie rod interacting with the loop, wherein the tie rod has a flexible end.

3. The device of claim 1 characterized in that the frame may have a conical or cylindrical shape.

4. The device of claim 1 characterized in that the rings have a zigzag or U-like shape with uniform or nonuniform arms.

5. The device of claim 1 characterized in that the interior coating can be made of a resorbable material.
